(19) United States
(12) Patent Application Publication
(10) Pub. No.: US 2025/0135230 A1
(43) Pub. Date: May 1, 2025

(54) Magnetic Resonance Imaging Guided Radiotherapy System and Magnetic Resonance Imaging Apparatus

(71) Applicant: OUR UNITED CORPORATION, Shaanxi (CN)

(72) Inventors: Hao Yan, Shaanxi (CN); Haifeng Liu, Shaanxi (CN)

(21) Appl. No.: 18/930,725

(22) Filed: Oct. 29, 2024

(30) Foreign Application Priority Data
Oct. 30, 2023 (CN) 202311423640.9
Oct. 30, 2023 (CN) 202322925957.4

(51) Int. Cl. A61N 5/10

(52) U.S. Cl. CPC A61N 5/1038; A61N 2005/1055

(57) ABSTRACT

A magnetic resonance imaging guided radiotherapy system and a magnetic resonance imaging apparatus. The radiotherapy system includes a rotating gantry, a treatment head, and a magnetic resonance imaging apparatus. The rotating gantry is rotatable about a preset rotation axis. The treatment head is rotatable along with rotation of the rotating gantry and configured to emit radiotherapy rays to a target object. The magnetic resonance imaging apparatus includes two magnet assemblies disposed opposite to each other on the rotating gantry in a circumferential direction of the rotating gantry and used to provide a magnetic field for imaging. The magnetic field for imaging has an overlapping region with the radiotherapy rays. A center axis of the magnet assemblies has a preset included angle with the rotation axis, and the closer to the center axis, the greater a distance between inner surfaces of the magnet assemblies disposed opposite to each other.

### CROSS-REFERENCE TO RELATED APPLICATIONS

This application claims priority to Chinese Patent Application No. 202311423640.9, filed on Oct. 30, 2023, and Chinese Patent Application No. 202322925957.4, filed on Oct. 30, 2023, which are hereby incorporated by reference in their entireties.

### BACKGROUND OF THE INVENTION

### Field of the Invention

The present disclosure relates to the technical field of radiotherapy, and in particular to a magnetic resonance imaging guided radiotherapy system and a magnetic resonance imaging apparatus.

### Description of Related Art

Compared with computed tomography (CT), magnetic resonance imaging (MRI) has an advantage that MRI images may clearly present the distribution of soft tissues, so that the position of tumor lesions may be more accurately determined. Therefore, MRI is increasingly widely applied to image guidance in radiotherapy, especially to real-time image guidance in radiotherapy.

### SUMMARY OF THE INVENTION

In an aspect, a magnetic resonance imaging guided radiotherapy system is provided. The radiotherapy system includes a rotating gantry, at least one treatment head, and a magnetic resonance imaging apparatus. The rotating gantry is rotatable about a preset rotation axis. The treatment head is rotatable along with rotation of the rotating gantry and configured to emit radiotherapy rays to a target object. The magnetic resonance imaging apparatus includes two magnet assemblies. The two magnet assemblies are disposed opposite to each other on the rotating gantry in a circumferential direction of the rotating gantry and used to provide a magnetic field for imaging. The magnetic field for imaging has an overlapping region with the radiotherapy rays, so as to perform magnetic resonance imaging on the target object. A center axis of the magnet assemblies disposed opposite to each other has a preset included angle with the rotation axis, and the closer to the center axis, the greater a distance between inner surfaces of the magnet assemblies disposed opposite to each other.

In another aspect, a magnetic resonance imaging apparatus is further provided in the present disclosure. The magnetic resonance imaging apparatus includes magnet assemblies disposed opposite to each other, and the magnet assemblies are used to provide a magnetic field for imaging. The magnet assemblies disposed opposite to each other have a center axis. Closer to the center axis, the greater a distance between inner surfaces of the magnet assemblies disposed opposite to each other.

### BRIEF DESCRIPTION OF THE DRAWINGS

In order to describe technical solutions in the embodiments of the present disclosure more clearly, the accompanying drawings to be used in description of embodiments will be introduced briefly. However, the accompanying drawings to be described below are merely some embodiments of the present disclosure, and a person of ordinary skill in the art may also obtain other accompanying drawings according to these accompanying drawings without paying any creative effort.

FIG. 1 is a schematic diagram showing a structure of a magnetic resonance imaging guided radiotherapy system, in accordance with some embodiments of the present disclosure;

FIG. 2 is a schematic diagram showing a structure of another magnetic resonance imaging guided radiotherapy system, in accordance with some embodiments of the present disclosure;

FIG. 3 is a schematic diagram showing a structure of yet another magnetic resonance imaging guided radiotherapy system, in accordance with some embodiments of the present disclosure;

FIG. 4 is a schematic diagram showing a structure of yet another magnetic resonance imaging guided radiotherapy system, in accordance with some embodiments of the present disclosure;

FIG. 5 is a top view of a first magnet assembly, in accordance with some embodiments of the present disclosure;

FIG. 6 is a schematic diagram showing a structure of yet another magnetic resonance imaging guided radiotherapy system, in accordance with some embodiments of the present disclosure;

FIG. 7 is a schematic diagram showing a structure of yet another magnetic resonance imaging guided radiotherapy system, in accordance with some embodiments of the present disclosure;

FIG. 8 is a schematic diagram showing a structure of yet another magnetic resonance imaging guided radiotherapy system, in accordance with some embodiments of the present disclosure;

FIG. 9 is a schematic diagram showing a structure of yet another magnetic resonance imaging guided radiotherapy system, in accordance with some embodiments of the present disclosure;

FIG. 10 is a schematic diagram showing a structure of yet another magnetic resonance imaging guided radiotherapy system, in accordance with some embodiments of the present disclosure;

FIG. 11 is a schematic diagram showing a structure of yet another magnetic resonance imaging guided radiotherapy system, in accordance with some embodiments of the present disclosure; and

FIG. 12 is a schematic diagram showing a structure of a magnetic resonance imaging apparatus, in accordance with some embodiments of the present disclosure.

### DESCRIPTION OF THE INVENTION

The technical solutions in the embodiments of the present disclosure will be clearly and completely described below with reference to the accompanying drawings in the embodiments of the present disclosure. However, the described embodiments are merely some but not all embodiments of the present disclosure. All other embodiments obtained by a person of ordinary skill in the art based on the embodiments of the present disclosure without paying any creative effort shall be included in the protection scope of the present disclosure.

In the description of the present disclosure, it will be understood that, orientations or positional relationships indicated by the terms such as “center,” “longitudinal,” “transverse,” “length,” “width,” “thickness,” “up,” “down,” “front,” “rear,” “left,” “right,” “vertical,” “horizontal,” “top,” “bottom,” “inner,” “outer,” and the like are based on orientations or positional relationships shown in the accompanying drawings, which are merely for convenience in description of the present disclosure and simplifying the description, but not to indicate or imply that the indicated devices or elements must have a specific orientation, or be constructed and operated in a specific orientation. Therefore, these terms will not be construed as limitations on the present disclosure.

Hereinafter, the terms such as “first” and “second” are used for descriptive purposes only and are not to be construed as indicating or implying the relative importance or implicitly indicating the number of indicated technical features. Thus, features defined by “first” or “second” may explicitly or implicitly include one or more of the features. In the description of the present disclosure, the term “a plurality of” or “the plurality of” means two or more unless otherwise expressly specified.

The phrase “at least one of A, B, and C” has the same meaning as the phrase “at least one of A, B, or C,” both including the following combinations of A, B, and C: only A, only B, only C, a combination of A and B, a combination of A and C, a combination of B and C, and a combination of A, B, and C.

The use of the phase “applicable to” or “configured to” herein means an open and inclusive expression, which does not exclude devices that are applicable to or configured to perform additional tasks or steps.

In the present disclosure, the term “exemplary” is used to mean “used as an example, instance or explanation.” Any embodiment described in the present disclosure as “exemplary” is not necessarily construed as being preferable or superior to other embodiments. The following description is given to enable any person skilled in the art to implement and use the present disclosure. Details are set forth for purposes of explanation in the following description. It will be understood that a person of ordinary skill in the art may recognize that the present disclosure may be implemented without using these specific details. In other embodiments, well-known structures and processes are not described in detail, so as to avoid obscuring the description of the present disclosure with unnecessary detail. Thus, the present disclosure is not intended to be limited by the embodiments shown, but is to be consistent with the broadest scope in accordance with the principles and features disclosed in the present disclosure.

Generally, a radiotherapy space or an imaging space of a radiotherapy system combined with a magnetic resonance imaging (MRI) apparatus is very small, which may cause physical discomfort to a patient located in the radiotherapy space or the imaging space.

In order to solve the above problems, a magnetic resonance imaging guided radiotherapy system and a magnetic resonance imaging apparatus are provided in some embodiments of the present disclosure, each of which is described in detail below.

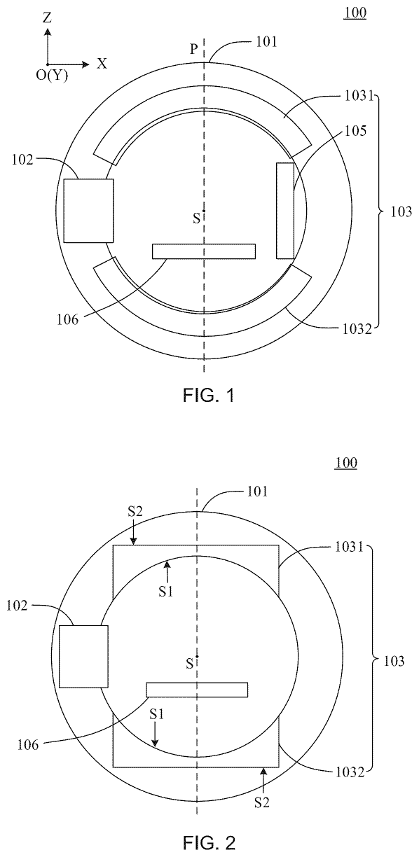

FIG. 1 is a schematic diagram showing a structure of a magnetic resonance imaging guided radiotherapy system, in accordance with some embodiments of the present disclosure. As shown in FIG. 1, the radiotherapy system 100 includes a rotating gantry 101, a treatment head 102, and a magnetic resonance imaging apparatus 103.

The rotating gantry 101 is rotatable about a preset rotation axis S. Here, the rotating gantry 101 may rotate clockwise around the preset rotation axis S, or may also rotate counterclockwise around the preset rotation axis S. Generally, a radiotherapy center of the radiotherapy system is located on the preset rotation axis S, and the preset rotation axis S is parallel to the Y axis in the International Electrotechnical Commission (IEC) coordinate system of the radiotherapy system.

In some embodiments, the rotating gantry 101 may be a ring gantry, a C-shaped arm gantry, a drum-shaped gantry, a gantry, or other types of gantries, and the present disclosure is not specifically limited thereto.

The treatment head 102 is configured to emit radiotherapy rays to a target object, and may be rotated with the rotation of the rotating gantry 101, so as to perform radiotherapy on a target or target volume of the target object. Here, the target object may be a patient or a phantom simulating a patient. The treatment head 102 may be directly disposed on the rotating gantry 101, or may also be indirectly connected to the rotating gantry 101. For example, the treatment head 102 is disposed on other devices connected to the rotating gantry 101.

There may be one treatment head 102 as shown in FIG. 1. In some embodiments, there may also be a plurality of treatment heads 102, such as two treatment heads, three treatment heads, or four treatment heads. The treatment head 102 may be a focused treatment head that focuses multiple radiotherapy rays into a single point (or the target), a conformal intensity-modulated treatment head that adjusts the radiotherapy rays into different shapes (or target volumes) according to a shape of the tumor, a pencil-shaped beam treatment head that restricts a radiotherapy ray into a pencil-shaped beam, or a treatment head of other radiotherapy modes.

A radiation source used by the treatment head 102 may be any radiation source. For example, the radiation source may be a particle accelerator (e.g., an X-ray accelerator) or a radioactive isotope source (e.g., a cobalt 60 radiation source). For example, the rays generated by the radiation source may be gamma rays, X-rays, electron rays, proton rays, helium ion rays, carbon ion rays, other heavy ion rays or neutron rays. For example, the treatment head 102 may be a gamma-ray focused treatment head, a gamma-ray conformal intensity-modulated treatment head, a gamma-ray pencil-shaped beam treatment head, an X-ray conformal intensity-modulated treatment head, an X-ray focused treatment head, or an X-ray pencil-shaped beam treatment head (i.e., an X-knife treatment head).

The magnetic resonance imaging apparatus 103 may include two magnet assemblies, namely a first magnet assembly 1031 and a second magnet assembly 1032. The two magnet assemblies are disposed opposite to each other on the rotating gantry 101 in a circumferential direction of the rotating gantry 101 and used to form a uniform magnetic field for imaging. The magnetic field for imaging has an overlapping region with the radiotherapy rays, so as to perform magnetic resonance imaging on the target object to generate a magnetic resonance image.

A center axis P (e.g., the dashed line P shown in FIG. 1) of the first magnet assembly 1031 and the second magnet assembly 1032 disposed opposite to each other may have a preset included angle with the rotation axis S. For example, the preset included angle is 90°. Moreover, the closer to the center axis P, the greater the distance between the inner surfaces of the magnet assemblies disposed opposite to each other, and correspondingly, the farther away from the center axis P, the less the distance between the inner surfaces of the magnet assemblies disposed opposite to each other.

It will be noted that the inner surface of the magnet assembly is a surface of the magnet assembly proximate to the rotation axis S. The present disclosure does not limit shapes of the inner surfaces of the magnet assemblies disposed opposite to each other, as long as the closer to the center axis P, the greater the distance between the inner surfaces of the magnet assemblies disposed opposite to each other, and the farther away from the center axis P, the less the distance between the inner surfaces of the magnet assemblies disposed opposite to each other.

For example, the inner surface of at least one of the magnet assemblies disposed opposite to each other is a curved surface or has a stepped shape or other shapes. Another surface of the magnet assembly away from the rotation axis S may be referred to as an outer surface of the magnet assembly, and a shape of the outer surface of the magnet assembly may be different from the shape of the inner surface of the magnet assembly. Here, the present disclosure does not limit the shape, curvature, size, etc. of the outer surface of the magnet assembly.

For example, as shown in FIG. 2, the inner surface S1 of at least one of the first magnet assembly 1031 or the second magnet assembly 1032 may be a smooth curved surface, and the outer surface S2 of at least one of the first magnet assembly 1031 or the second magnet assembly 1032 is a horizontal surface; the inner surface S1 of the second magnet assembly 1032 disposed opposite to the first magnet assembly 1031 may be a smooth curved surface, and the outer surface S2 of the second magnet assembly 1032 is a horizontal surface. Of course, the outer surface S2 of the second magnet assembly 1032 may also be a smooth curved surface.

It will also be noted that the magnet assemblies (e.g., the first magnet assembly 1031 and the second magnet assembly 1032) disposed opposite to each other may be symmetrical with respect to a direction of the center axis P. Generally, each magnet assembly has a regular shape, and the center axis P is also a center axis of each magnet assembly.

Here, a magnetic field strength generated by the magnet assemblies disposed opposite to each other may be within a range of 0.2 T (Tesla) to 3 T, inclusive, such as 0.35 T or 1.0 T. Magnets in the magnet assemblies disposed opposite to each other may be permanent magnets, conventional electromagnets or superconducting magnets, etc. If the magnet assemblies disposed opposite to each other are permanent magnets, they may generally have a magnetic field strength of 0.2 T to 1.0 T. If the magnet assemblies disposed opposite to each other are superconducting magnets, they may generally have a magnetic field strength of 1.5 T to 3.0 T, such as 1.5 T or 3.0 T.

In some embodiments of the present disclosure, in a case where the MRI guided radiotherapy system is used to perform MRI guided radiotherapy, the magnetic field formed by the magnet assemblies disposed opposite to each other in the MRI apparatus may be used to perform nuclear magnetic resonance imaging on the target object to be treated. The target object is guided to a position to be treated based on the magnetic resonance image generated by nuclear magnetic resonance imaging, and then the target object is subjected to radiotherapy using the treatment head. Alternatively, the position of the target object is monitored in real time based on the magnetic resonance image generated by nuclear magnetic resonance imaging during the process of the treatment head performing radiotherapy on the target object.

Since the closer to the center axis of the magnet assemblies disposed opposite to each other, the greater the distance between the inner surfaces of the magnet assemblies disposed opposite to each other, the size of the imaging space and the treatment space of the radiotherapy system is increased, thereby reducing or alleviating the discomfort of the target object located in the imaging or treatment space.

For the magnetic resonance imaging apparatus 103 in the radiotherapy system 100, at least one (e.g., the first magnet assembly 1031 or the second magnet assembly 1032) of the magnet assemblies disposed opposite to each other may be a whole magnet as shown in FIG. 1 or 2. In some embodiments, at least one of the magnet assemblies disposed opposite to each other may further include a plurality of magnets, and the plurality of magnets are arranged in sequence in the direction of the center axis P.

In some embodiments, the plurality of magnets may be nested and arranged in sequence along the direction of the center axis P, and may be staggered with each other, and the staggered positions are not limited. For example, side surfaces of the plurality of magnets are arranged in a staggered manner in the direction of the center axis P; alternatively, upper surfaces and lower surfaces of the plurality of magnets are arranged in a staggered manner in a direction perpendicular to the center axis P; alternatively, the side surfaces of the plurality of magnets are arranged in a staggered manner in the direction of the center axis P, and the upper surfaces and lower surfaces of the plurality of magnets are arranged in a staggered manner in the direction perpendicular to the center axis P.

Of course, the side surfaces or the upper surfaces and lower surfaces may also be arranged in a non-staggered manner, the side surfaces are only arranged in the direction of the center axis P, and the upper surfaces and lower surfaces are only arranged in the direction perpendicular to the center axis P. For example, the side surfaces of the plurality of magnets are arranged in a non-staggered manner in the direction of the center axis P, and the upper and lower surfaces of the plurality of magnets are arranged in a non-staggered manner in the direction perpendicular to the center axis P.

Here, inner surfaces of the plurality of magnets constitute the inner surface of the corresponding magnet assembly. The inner surface of the magnet assembly may be a smooth curved surface as shown in FIG. 1 or 2, or may also have a stepped shape or other shapes. In addition, a thickness of each of the plurality of magnets may be same. Of course, the thickness of at least one of the plurality of magnets may be different from that of other magnets. Ring widths of annular magnets in the plurality of magnets may be same or different.

It will be noted that the thickness of the magnet is a thickness of a section of the magnet viewed from the preset rotation axis S (or the Y axis), and the ring width of the annular magnet is a ring width of the upper surface (or the lower surface) of the annular magnet viewed from the center axis P.

It will also be noted that, the magnetic field strength of the magnetic field formed by each group of magnets disposed oppositely may be same in the plurality of magnets disposed oppositely in the magnet assemblies disposed oppositely. Of course, the magnetic field strength of the magnetic field formed by at least one group of magnets disposed oppositely may also be different from the magnetic field strength of the magnetic field formed by other magnets disposed oppositely.

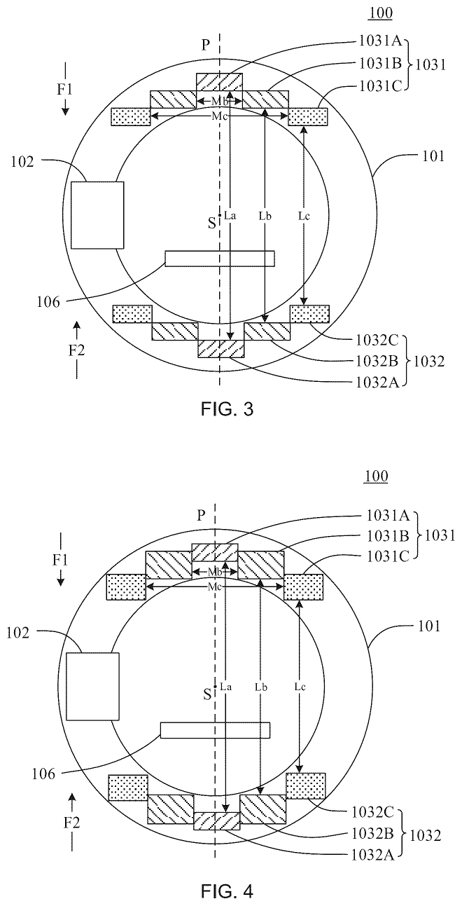

For example, as shown in FIGS. 3 and 4, the magnet assemblies disposed oppositely in the magnetic resonance imaging apparatus 103 include a first magnet assembly 1031 and a second magnet assembly 1032. The first magnet assembly 1031 includes a first magnet 1031A, a second magnet 1031B, and a third magnet 1031C that are nested and arranged in sequence in a direction (e.g., a first direction F1) of the center axis P, and the second magnet assembly 1032 includes a fourth magnet 1032A, a fifth magnet 1032B, and a sixth magnet 1032C that are nested and arranged in sequence in a direction (e.g., a second direction F2 opposite to the first direction F1) of the center axis P.

The fourth magnet 1032A, the fifth magnet 1032B, and the sixth magnet 1032C are disposed opposite to the first magnet 1031A, the second magnet 1031B, and the third magnet 1031C, respectively, thereby forming the magnetic field for imaging. The magnetic field strength of the magnetic field formed by the first magnet 1031A and the fourth magnet 1032A, the magnetic field strength of the magnetic field formed by the second magnet 1031B and the fifth magnet 1032B, and the magnetic field strength of the magnetic field formed by the third magnet 1031C and the sixth magnet 1032C are all the same.

The inner surface of the first magnet assembly 1031 formed by inner surfaces of the first magnet 1031A, the second magnet 1031B, and the third magnet 1031C, and the inner surface of the second magnet assembly 1032 formed by inner surfaces of the fourth magnet 1032A, the fifth magnet 1032B, and the sixth magnet 1032C both have a stepped shape.

It may be seen from FIG. 3 or 4 that a distance La between the inner surfaces of the first magnet 1031A and the fourth magnet 1032A disposed opposite to each other is greater than a distance Lb between the inner surfaces of the second magnet 1031B and the fifth magnet 1032B disposed opposite to each other, and the distance Lb between the inner surfaces of the second magnet 1031B and the fifth magnet 1032B disposed opposite to each other is greater than a distance Lc between the inner surfaces of the third magnet 1031C and the sixth magnet 1032C disposed opposite to each other.

Considering the first magnet assembly 1031 as an example, in FIG. 3, a side surface of the first magnet 1031A and a side surface of the second magnet 1031B are not staggered, and a side surface of the second magnet 1031B and a side surface of the third magnet 1031C are not staggered, and these side surfaces are only arranged in the direction (e.g., the first direction F1) of the center axis P. However, the side surface of the first magnet 1031A and the side surface of the second magnet 1031B are staggered in FIG. 4.

For example, an inner side surface of the second magnet 1031B proximate to the center axis P is approximately located at one-half of the side surface of the first magnet 1031A. Similarly, the side surface of the second magnet 1031B and the side surface of the third magnet 1031C are also staggered. For example, an inner side surface of the third magnet 1031C proximate to the center axis P is approximately located at one fifth of an outer side surface of the second magnet 1031B away from the center axis P.

In addition, the thicknesses of the first magnet 1031A, the second magnet 1031B, and the third magnet 1031C are same in FIG. 3. However, the thicknesses of the first magnet 1031A, the second magnet 1031B, and the third magnet 1031C are different in FIG. 4. For example, the second magnet 1031B is the thickest and the first magnet 1031A is the thinnest.

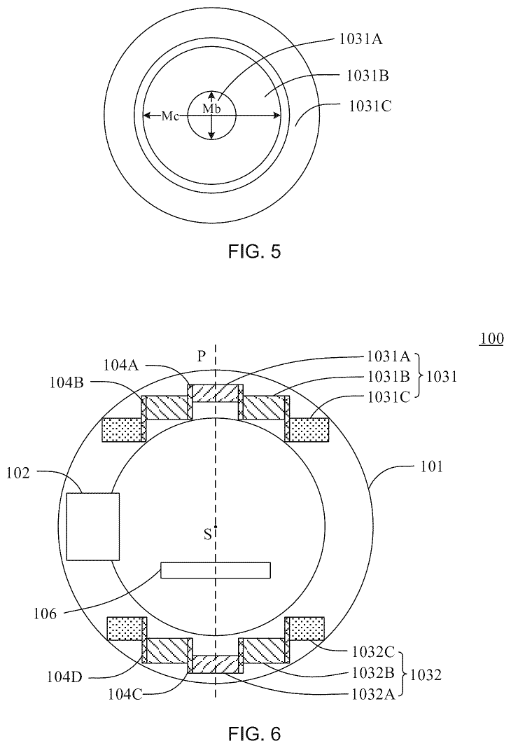

With reference to FIG. 5 based on FIGS. 3 and 4, the first magnet 1031A in the first magnet assembly 1031 is a cylinder without a through hole, the second magnet 1031B and the third magnet 1031C are annular cylinders, and diameters of center through holes of the second magnet 1031B and the third magnet 1031C are Mb and Mc, respectively, and Mc is greater than Mb. In this way, the first magnet assembly 1031 formed by the first magnet 1031A, the second magnet 1031B, and the third magnet 1031C has no through hole in the direction of the center axis P.

If a diameter of a center through hole of the first magnet 1031A is regarded as 0, the first magnet 1031A, the second magnet 1031B, and the third magnet 1031C are arranged along the first direction F1 of the center axis P, namely a direction toward the rotation axis S, in order of the center through holes from small to large. Correspondingly, the fourth magnet 1032A, the fifth magnet 1032B, and the sixth magnet 1032C in the second magnet assembly 1032 are arranged along the second direction F2 of the center axis P opposite to the first direction F1, namely a direction toward the rotation axis S, in order of the center through holes from small to large.

In addition, as shown in FIG. 5, the ring widths of the annular second magnet 1031B and the annular third magnet 1031C (or the fifth magnet 1032B and the sixth magnet 1032C) are same viewed from the Z axis direction (e.g., the direction of the center axis P), but the ring widths may also be different.

In a case where the plurality of magnets are arranged in sequence along the direction of the center axis P, the radiotherapy system 100 may further include a transition member, and the transition member may be disposed between at least two of the plurality of magnets. The transition member is made of non-magnetic materials, such as porcelain, colored glaze, enamel, and non-ferromagnetic materials, which cannot be magnetized. In this way, a relatively uniform magnetic field is formed.

Here, the transition member made of non-magnetic material may be an annular cylinder, and the ring width of the annular shape and the thickness of the cylinder are not specifically limited in the present disclosure.

For example, as shown in FIG. 6, a first transition member 104A is disposed between the first magnet 1031A and the second magnet 1031B, a second transition member 104B is disposed between the second magnet 1031B and the third magnet 1031C, a third transition member 104C is disposed between the fourth magnet 1032A and the fifth magnet 1032B, and a fourth transition member 104D is disposed between the fifth magnet 1032B and the sixth magnet 1032C.

As shown in FIGS. 1 to 4 and 6, each of the magnet assemblies disposed opposite to each other does not have a through hole in the direction of the center axis P. However, in some embodiments, at least one of the magnet assemblies disposed opposite to each other may have a through hole in the direction of the center axis P, so that other devices such as the treatment head may be easily installed.

It will be noted that, the treatment head 102 may be located between the magnet assemblies disposed opposite to each other regardless of whether at least one of the magnet assemblies has the through hole in the direction of the center axis P. Here, the treatment head 102 located between the magnet assemblies disposed opposite to each other may be the focused treatment head, the conformal intensity-modulated treatment head, the pencil-shaped beam treatment head, or the treatment head of other radiotherapy modes.

Moreover, the radiation source used by the treatment head may be any radiation source, and the radiation source may generate gamma rays, electron rays, proton rays, helium ion rays, carbon ion rays, other heavy ion rays, neutron rays, or other non-X-rays. In a case where the treatment head is a gamma-ray treatment head, the treatment head may further be a gamma-ray focused treatment head or a gamma-ray conformal intensity-modulated treatment head.

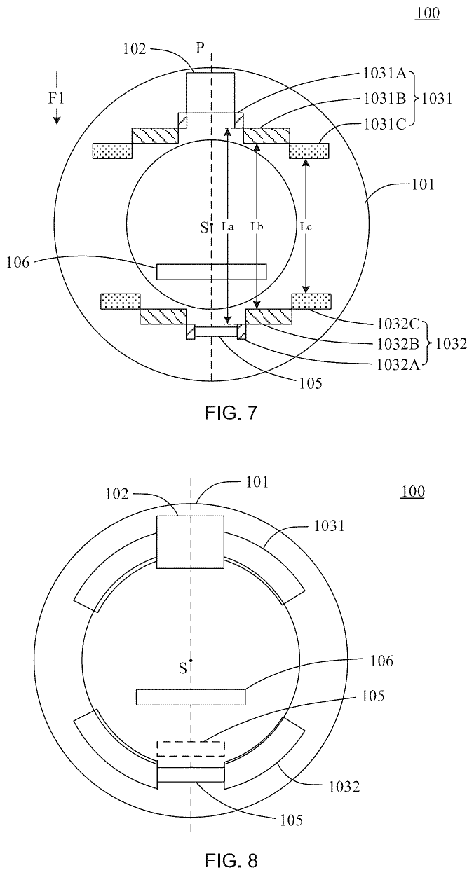

If at least one of the magnet assemblies disposed opposite to each other has the through hole in the direction of the center axis P, as shown in FIGS. 7 and 8, the first magnet 1031A and the fourth magnet 1032A whose inner surfaces are farthest apart in the magnet assemblies disposed opposite to each other have through holes, the treatment head 102 may be located at the through hole of the first magnet 1031A or the fourth magnet 1032A, and the radiotherapy rays emitted by the treatment head 102 are used to irradiate the target object.

Here, the treatment head 102 located at the through hole may be a focused treatment head, a conformal intensity-modulated treatment head, a pencil-shaped beam treatment head, or a treatment head of other radiotherapy modes. Moreover, the radiation source used by the treatment head 102 may be any radiation source. For example, the radiation source is a radiation source emitting rays such as gamma rays, X-rays, electrons rays, protons rays, helium ion rays, carbon ion rays, other heavy ion rays, or neutron rays.

It will be noted that the treatment head 102 located at the through hole may mean that the treatment head 102 is located above the through hole as shown in FIG. 7, or the treatment head 102 runs through the through hole as shown in FIG. 8, or the treatment head 102 may also be located inside the through hole or at other positions.

In some embodiments, the radiotherapy system 100 may further include a detector 105. As shown in FIGS. 1, 7 and 8, the detector 105 may be disposed at a position opposite to the treatment head 102, and the detector 105 is used to receive the radiotherapy rays emitted by the treatment head 102 and passing through the target object and generate corresponding image data.

As shown in FIGS. 7 and 8, in a case where the first magnet assembly 1031 and the second magnet assembly 1032 both have through holes, that is, in a case where the first magnet 1031A and the fourth magnet 1032A both have through holes, the treatment head 102 may be located at the through hole of the first magnet 1031A in the first magnet assembly 1031, and correspondingly, the detector 105 may be located at the through hole of the fourth magnet 1032A in the second magnet assembly 1032.

It will be noted that the detector 105 located at the through hole may mean that the detector 105 is located inside the through hole as shown in FIGS. 7 and 8, or the detector 105 may also be located above the through hole, or run through the through hole, or at other positions.

In some embodiments, the detector 105 may further have a driving device. The driving device may drive the detector 105 to move. For example, as shown in FIG. 8, the driving device drives the detector 105 close to or away from the treatment head 102 (that is, the driving device drives the detector 105 to move up and down or move along the Z axis direction). Of course, the driving device may also drive the detector 105 to move left and right (that is, the driving device drives the detector 105 to move along the X axis direction) or forward and backward (that is, the driving device drives the detector 105 to move along the Y axis direction).

In some embodiments, the radiotherapy system 100 may further include a patient support device 106. The patient support device 106 may support and position the target object. For example, the patient support device 106 may be a three-dimensional, four-dimensional, five-dimensional, or six-dimensional patient support device or a treatment chair. The patient support device 106 is located in the imaging space or the radiotherapy space of the radiotherapy system and may be moved into or out of the overlapping region in the direction of the rotation axis S. Of course, the patient support device 106 may also move in other directions.

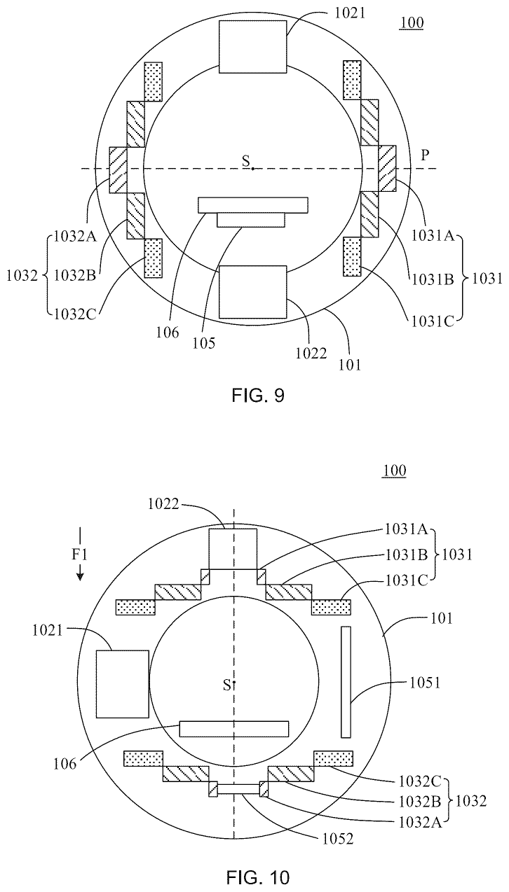

In some embodiments, the detector 105 may be connected to the patient support device 106 and move along with the movement of the patient support device 106, and the detector 105 may be disposed under the patient support device 106. For example, the detector 105 may be disposed on a lower surface of a couch board of the patient support device 106 as shown in FIG. 9, or may also be disposed inside a couch body of the patient support device 106.

Here, the detector 105 may be a megavolt (MV) level detector, such as an electronic portal imaging device (EPID), or may also be a kilovolt (KV) level detector. The detector 105 may be a flat-panel detector or an arc-shaped detector.

The treatment head 102 in the radiotherapy system 100 may be one shown in FIG. 1, FIG. 2, FIG. 3, FIG. 4, FIG. 6, or FIG. 7. In some embodiments, there may also be a plurality of treatment heads 102, and the plurality of treatment heads 102 include a first treatment head and a second treatment head. Here, the first treatment head and the second treatment head may emit radiotherapy rays simultaneously, or may emit radiotherapy rays separately.

It will be noted that, if there are the plurality of treatment heads 102, the plurality of treatment heads 102 may be treatment heads of a same radiotherapy mode or treatment heads of different radiotherapy modes. For example, the treatment head located between the magnet assemblies disposed opposite to each other may be a gamma-ray treatment head, and the treatment head located at the through hole may be a gamma-ray treatment head or an X-ray treatment head. The radiation source used by each treatment head 102 may be same or different.

In some embodiments, a detector may be disposed at a position opposite to the first treatment head; alternatively, a detector may be disposed at a position opposite to the second treatment head; alternatively, a detector may be disposed at a position opposite to the first treatment head, and a detector may be disposed at a position opposite to the second treatment head.

The arrangement structure between the plurality of treatment heads and the magnetic resonance imaging apparatus in the radiotherapy system will be described below by some specific examples.

In an example, as shown in FIG. 9, the treatment heads 102 include a first treatment head 1021 and a second treatment head 1022. The first treatment head 1021 and the second treatment head 1022 are located on two sides between the first magnet assembly 1031 and the second magnet assembly 1032 disposed opposite to each other, respectively.

Here, the first treatment head 1021 and the second treatment head 1022 may be treatment heads of different modes, and the radiation sources of the first treatment head 1021 and the second treatment head 1022 may be same or different. In some examples, the first treatment head 1021 and the second treatment head 1022 located between the magnet assemblies disposed opposite to each other may be gamma-ray treatment heads. For example, the first treatment head 1021 is a gamma-ray focused treatment head, and the second treatment head 1022 is a gamma-ray conformal intensity-modulated treatment head.

In this example, the detector 105 may be disposed on the lower surface of the patient support device 106. When the rotating gantry 101 rotates and drives at least one of the first treatment head 1021 or the second treatment head 1022 to be opposite to the detector 105, the detector 105 may obtain at least one of the radiotherapy rays passing through the target object or the radiotherapy rays emitted by the first treatment head 1021 or the second treatment head 1022, and generate corresponding image data. The image data may reflect the radiation dose received by the target object during the radiotherapy process.

It will be noted that when the first treatment head 1021 and the second treatment head 1022 emit radiotherapy rays at the same time, two surfaces of the detector 105 may simultaneously obtain the radiotherapy rays and generate image data.

In another example, as shown in FIG. 10, the treatment heads 102 include a first treatment head 1021 and a second treatment head 1022. The first treatment head 1021 is located between the first magnet assembly 1031 and the second magnet assembly 1032 disposed opposite to each other, and the second treatment head 1022 is located at a through hole of the first magnet assembly 1031. The radiotherapy rays emitted by the first treatment head 1021 and the second treatment head 1022 irradiate the target object.

Furthermore, a detector may be disposed at a position opposite to at least one of the first treatment head 1021 or the second treatment head 1022. In this example, the radiotherapy system 100 may include a plurality of detectors 105, such as a first detector 1051 and a second detector 1052. The first detector 1051 is disposed at a position opposite to the first treatment head 1021, and the second detector 1052 is disposed at a position opposite to the second treatment head 1022. The first detector 1051 and the second detector 1052 each are used to obtain the radiotherapy rays passing through the target object. Of course, there may be only the first detector 1051 disposed at the position opposite to the first treatment head 1021, or there may be only the second detector 1052 disposed at the position opposite to the second treatment head 1022.

Here, the first treatment head 1021 and the second treatment head 1022 may be treatment heads of different modes, and the radiation sources of the first treatment head 1021 and the second treatment head 1022 may be same or different. In some examples, the first treatment head 1021 located between the magnet assemblies disposed opposite to each other may be a gamma-ray treatment head, such as a gamma-ray focused treatment head. The second treatment head 1022 located at the through hole of the first magnet assembly 1031 is an X-ray treatment head, such as an X-ray conformal intensity-modulated treatment head.

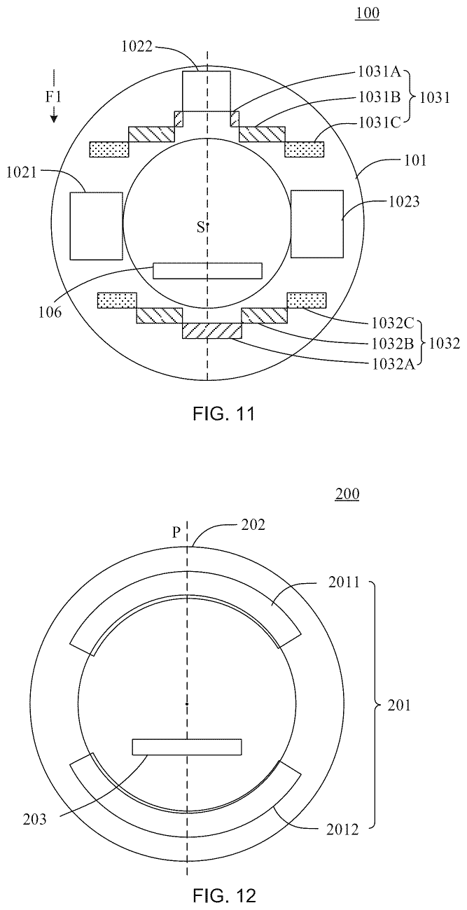

It may be understood that the radiotherapy system may include more treatment heads, such as, three treatment heads. As shown in FIG. 11, the treatment heads 102 include a first treatment head 1021, a second treatment head 1022, and a third treatment head 1023. The first treatment head 1021 and the third treatment head 1023 are located on two sides between the first magnet assembly 1031 and the second magnet assembly 1032 disposed opposite to each other, and the second treatment head 1022 is located at a through hole of the first magnet assembly 1031.

Here, the first treatment head 1021 may be a gamma-ray focused treatment head, the second treatment head 1022 may be an X-ray conformal intensity-modulated treatment head, and the third treatment head 1023 may be a gamma-ray conformal intensity-modulated treatment head.

Of course, the radiotherapy system may include four, five or more treatment heads, and the present disclosure is not limited thereto. The treatment head located between the magnet assemblies disposed opposite to each other may be a gamma-ray treatment head, and the treatment head located at the through hole may be a gamma-ray treatment head or an X-ray treatment head.

FIG. 12 shows a magnetic resonance imaging apparatus provided in some embodiments of the present disclosure. As shown in FIG. 12, the magnetic resonance imaging apparatus 200 includes magnet assemblies 201 disposed opposite to each other, namely, a first magnet assembly 2011 and a second magnet assembly 2012, for forming a uniform magnetic field for imaging. The magnet assemblies 201 disposed opposite to each other have a center axis P. The closer to the center axis P, the greater the distance between inner surfaces of the magnet assemblies disposed opposite to each other, and the farther away from the center axis P, the less the distance between the inner surfaces of the magnet assemblies disposed opposite to each other.

Furthermore, the magnetic resonance imaging apparatus 200 may further include a gantry 202, and the magnet assemblies 201 disposed opposite to each other are disposed on the gantry 202.

The magnetic resonance imaging apparatus 200 may further include a patient support device 203 for supporting a target object.

It will be noted that the arrangement of the magnet assemblies 201 disposed opposite to each other is the same as that of the magnet assemblies disposed opposite to each other in the magnetic resonance imaging apparatus 103, and details will not be repeated herein.

When the magnetic resonance imaging apparatus in the embodiments of the present disclosure performs magnetic resonance imaging, the magnetic resonance imaging apparatus uses the magnetic field formed by the magnet assemblies disposed opposite to each other to perform nuclear magnetic resonance imaging on the target object to be treated, so as to generate a nuclear magnetic resonance image. Since the closer to the center axis of the magnet assemblies disposed opposite to each other, the greater the distance between the inner surfaces of the magnet assemblies disposed opposite to each other, the imaging space of the magnetic resonance imaging apparatus is increased, thereby reducing or alleviating the discomfort of the target object located in the imaging or treatment space.

The magnetic resonance imaging guided radiotherapy system and the magnetic resonance imaging apparatus provided in some embodiments of the present disclosure are described above in detail. Specific examples are used herein to expound the principles and implementations of the present disclosure. The description of the above embodiments is only used to help understand the method and core idea of the present disclosure. Meanwhile, for those skilled in the art, there will be changes in the specific implementations and application scope according to the idea of the present disclosure. In summary, the content of this specification will not be understood as a limitation on the present disclosure.

What is claimed is:

1. A magnetic resonance imaging guided radiotherapy system, comprising:
a rotating gantry rotatable about a preset rotation axis;
at least one treatment head rotatable along with rotation of the rotating gantry and configured to emit radiotherapy rays to a target object; and
a magnetic resonance imaging apparatus including two magnet assemblies, the two magnet assemblies being disposed opposite to each other on the rotating gantry in a circumferential direction of the rotating gantry and used to provide a magnetic field for imaging, the magnetic field for imaging having an overlapping region with the radiotherapy rays, so as to perform magnetic resonance imaging on the target object; wherein a center axis of the magnet assemblies disposed opposite to each other has a preset included angle with the rotation axis, and closer to the center axis, greater a distance between inner surfaces of the magnet assemblies disposed opposite to each other.

2. The system according to claim 1, wherein the at least one treatment head satisfies one of followings:
the at least one treatment head is located between the magnet assemblies disposed opposite to each other; and
at least one of the magnet assemblies disposed opposite to each other has a through hole in a direction of the center axis, and correspondingly, the at least one treatment head is located at the through hole, and the radiotherapy rays emitted by the at least one treatment head irradiate the target object.

3. The system according to claim 2, wherein the at least one treatment head includes a first treatment head and a second treatment head, and the first treatment head and the second treatment head are located on two sides between the magnet assemblies disposed opposite to each other, respectively.

4. The system according to claim 2, wherein the at least one treatment head includes a first treatment head and a second treatment head, the first treatment head is located between the two magnet assemblies disposed opposite to each other, the second treatment head is located at the through hole, and the radiotherapy rays emitted by the first treatment head and the second treatment head irradiate the target object.

5. The system according to claim 2, wherein the treatment head located between the magnet assemblies disposed opposite to each other is a gamma-ray treatment head, and the treatment head located at the through hole is a gamma-ray treatment head or an X-ray treatment head.

6. The system according to claim 1, wherein any one of the magnet assemblies disposed opposite to each other includes a plurality of magnets, and the plurality of magnets are arranged in sequence in a direction of the center axis.

7. The system according to claim 6, wherein the plurality of magnets are nested and arranged.

8. The system according to claim 7, wherein the plurality of magnets are arranged in a staggered manner.

9. The system according to claim 6, wherein at least one of the plurality of magnets has a different thickness than other magnets.

10. The system according to claim 6, wherein the plurality of magnets of one of the two magnet assemblies are disposed opposite to the plurality of magnets of another of the two magnet assemblies, respectively.

11. The system according to claim 6, further comprising a transition member, the transition member being disposed between at least two magnets of the plurality of magnets, and the transition member being made of a non-magnetic material.

12. The system according to claim 1, wherein the inner surface of at least one of the magnet assemblies disposed opposite to each other is a curved surface or has a stepped shape.

13. The system according to claim 1, wherein a magnetic field strength generated by the magnet assemblies disposed opposite to each other is within a range of 0.2 T to 3 T, inclusive.

14. The system according to claim 1, further comprising at least one detector, the at least one detector being disposed at a position opposite to the at least one treatment head and configured to receive the radiotherapy rays emitted by the at least one treatment head and generate corresponding image data.

15. The system according to claim 14, wherein the at least one detector includes a plurality of detectors, the at least one treatment head includes a plurality of treatment heads, the plurality of detectors correspond to the plurality of treatment heads, respectively, and any one of the plurality of detectors is disposed at a position opposite to the corresponding treatment head.

16. The system according to claim 14, wherein in a case where both of the two magnet assemblies have through holes and the at least one treatment head is located at the through hole of one of the two magnet assemblies, the at least one detector is located at the through hole of another of the two magnet assemblies.

17. The system according to claim 14, further comprising a patient support device, the patient support device being capable of moving into or out of the overlapping region in a direction of the rotation axis, and the detector being connected to the patient support device and capable of moving with movement of the patient support device.

18. A magnetic resonance imaging apparatus, comprising:
magnet assemblies disposed opposite to each other and used to provide a magnetic field for imaging;
wherein the magnet assemblies disposed opposite to each other have a center axis, and closer to the center axis, greater a distance between inner surfaces of the magnet assemblies disposed opposite to each other.

19. The magnetic resonance imaging apparatus according to claim 18, wherein any one of the magnet assemblies disposed opposite to each other includes a plurality of magnets, and the plurality of magnets are arranged in sequence in a direction of the center axis.

20. The magnetic resonance imaging apparatus according to claim 18, wherein the inner surface of at least one of the magnet assemblies disposed opposite to each other is a curved surface or has a stepped shape.
